SEQUENCE LISTING (7 sequences)

SEQ ID NO: 1 - protein, 339 aa, Homo sapiens
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNLVRICLGAVILIILAGFLAEDWHSRRKRLRHRGRAVQRPLPPLPPLPQTRKSHGGQDGGRQDVHSRGLCS

SEQ ID NO: 2 - DNA, 1020 bp, Homo sapiens
atgtctccatccccgaccgccctcttctgtcttgggctgtgtctggggcgtgtgccagcgcagagtggaccgctccccaagccctccctccaggctctgcccagctccctggtgcccctggagaagccagtgaccctccggtgccagggacctccgggcgtggacctgtaccgcctggagaagctgagttccagcaggtaccaggatcaggcagtcctcttcatcccggccatgaagagaagtctggctggacgctaccgctgctcctaccagaacggaagcctctggtccctgcccagcgaccagctggagctcgttgccacgggagtttttgccaaaccctcgctctcagcccagcccggcccggcggtgtcgtcaggaggggacgtaaccctacagtgtcagactcggtatggctttgaccaatttgctctgtacaaggaaggggaccctgcgccctacaagaatcccgagagatggtaccgggctagtttccccatcatcacggtgaccgccgcccacagcggaacctaccgatgctacagcttctccagcagggacccatacctgtggtcggcccccagcgaccccctggagcttgtggtcacaggaacctctgtgacccccagccggttaccaacagaaccaccttcctcggtagcagaattctcagaagccaccgctgaactgaccgtctcattcacaaacaaagtcttcacaactgagacttctaggagtatcaccaccagtccaaaggagtcagactctccagctggtcctgcccgccagtactacaccaagggcaacctggtccggatatgcctcggggctgtgatcctaataatcctggcggggtttctggcagaggactggcacagccggaggaagcgcctgcggcacaggggcagggctgtgcagaggccgcttccgcccctcccgcccctcccgcagacccggaaatcacacgggggtcaggatggaggccgacaggatgttcacagccgcgggttatgttcatga

SEQ ID NO: 3 - protein, 247 aa, Homo sapiens
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTK

SEQ ID NO: 4 - protein, 913 aa, Homo sapiens
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 5 - protein, 398 aa, Homo sapiens
DADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPT

SEQ ID NO: 6 - protein, 855 aa, Homo sapiens
MILIPRMLLVLFLLLPILSSAKAQVNPAICRYPLGMSGGQIPDEDITASSQWSESTAAKYGRLDSEEGDGAWCPEIPVEPDDLKEFLQIDLHTLHFITLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSYNAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNMFAKGVKIFKEVQCYFRSEASEWEPNAISFPLVLDDVNPSARFVTVPLHHRMASAIKCQYHFADTWMMFSEITFQSDAAMYNNSEALPTSPMAPTTYDPMLKVDDSNTRILIGCLVAIIFILLAIIVIILWRQFWQKMLEKASRRMLDDEMTVSLSLPSDSSMFNNNRSSSPSEQGSNSTYDRIFPLRPDYQEPSRLIRKLPEFAPGEEESGCSGVVKPVQPSGPEGVPHYAEADIVNLQGVTGGNTYSVPAVTMDLLSGKDVAVEEFPRKLLTFKEKLGEGQFGEVHLCEVEGMEKFKDKDFALDVSANQPVLVAVKMLRADANKNARNDFLKEIKIMSRLKDPNIIHLLSVCITDDPLCMITEYMENGDLNQFLSRHEPPNSSSSDVRTVSYTNLKFMATQIASGMKYLSSLNFVHRDLATRNCLVGKNYTIKIADFGMSRNLYSGDYYRIQGRAVLPIRWMSWESILLGKFTTASDVWAFGVTLWETFTFCQEQPYSQLSDEQVIENTGEFFRDQGRQTYLPQPAICPDSVYKLMLSCWRRDTKNRPSFQEIHLLLLQQGDE

SEQ ID NO: 7 - protein, 378 aa, Homo sapiens
KAQVNPAICRYPLGMSGGQIPDEDITASSQWSESTAAKYGRLDSEEGDGAWCPEIPVEPDDLKEFLQIDLHTLHFITLVGTQGRHAGGHGIEFAPMYKINYSRDGTRWISWRNRHGKQVLDGNSNPYDIFLKDLEPPIVARFVRFIPVTDHSMNVCMRVELYGCVWLDGLVSYNAPAGQQFVLPGGSIIYLNDSVYDGAVGYSMTEGLGQLTDGVSGLDDFTQTHEYHVWPGYDYVGWRNESATNGYIEIMFEFDRIRNFTTMKVHCNNMFAKGVKIFKEVQCYFRSEASEWEPNAISFPLVLDDVNPSARFVTVPLHHRMASAIKCQYHFADTWMMFSEITFQSDAAMYNNSEALPTSPMAPTTYDPMLKVDDSNTR

(19) United States
(12) Patent Application Publication
(10) Pub. No.: US 2010/0068145 A1
(43) Pub. Date: Mar. 18, 2010

(54) Bispecific fusion protein having therapeutic and diagnostic potential

(75) Inventors: Harald Langer, Tuebingen (DE); Meinrad Gawaz, Tuebingen (DE); Hans-Jorg Buhring, Tuebingen (DE); Thomas Skutella, Tuebingen (DE); Gundram Jung, Rottenburg-Wendelsheim (DE)

Correspondence Address: MORRISON & FOERSTER LLP, 12531 HIGH BLUFF DRIVE, SUITE 100, SAN DIEGO, CA, 92130-2040, US

(73) Assignee: Eberhard-Karls-Universitaet Tuebingen, Tuebingen (DE)

(21) Appl. No.: 12/583,506

(22) Filed: Aug. 20, 2009

Related U.S. Application Data
(63) Continuation of application No. PCT/EP2008/001369, filed on Feb. 21, 2008.

(30) Foreign Application Priority Data
Feb. 22, 2007 (DE) 10 2007 010 306.0

(51) Int. Cl. A61K 49/00; C07K 14/00; C07K 16/00; C07H 21/04; A61K 39/395; C07K 1/00; A61P 9/10

(52) U.S. Cl. 424/9.1; 530/350; 530/387.3; 536/23.4; 424/136.1; 530/402

(57) ABSTRACT

The present invention relates to a bispecific fusion protein, comprising (a) a first polypeptide which binds to collagen, and (b) a second polypeptide which binds to endothelial precursor cells. Also, pharmaceutical compositions are disclosed, comprising the fusion protein of the invention, as well as methods for using the fusion protein, in particular for treating or preventing lesions of vessels and tissues.

### RELATED APPLICATION

This application is a continuation of copending International Patent Application PCT/EP2008/001369 filed on Feb. 21, 2008 and designating the United States, which was not published in English, and claims priority of German Patent Application DE 10 2007 010 306.0, filed on Feb. 22, 2007, both of which are incorporated herein by reference in their entireties.

### BACKGROUND OF THE INVENTION

The invention relates to a bispecific fusion protein having therapeutic and diagnostic potential for treatment/diagnosis of lesions of vessels or tissues; the invention furthermore relates to a nucleic acid molecule encoding this fusion protein, a pharmaceutical and diagnostic composition which comprises the fusion protein or nucleic acid molecule encoding therefore, and a method for using the bispecific fusion protein or nucleic acid molecule for treatment or prevention of lesions of vessels/tissues of a mammalian subject and a method for therapy of acute or chronic vascular diseases.

Damage to the vessels of the cardiovascular system occurs in particular as a consequence of stent or stent graft implants into the vessels, which in turn have to be inserted into the vessels affected because of other diseases or events in order to ensure supply of the surrounding tissue or to organs.

In the physiological state, the blood circulates in a closed system of vessels without the flow of blood ceasing or blood exiting into surrounding tissue. Needless to say, damage in the vessel wall leads to the integrity of the vessel wall being eliminated and to subsequent hemorrhaging into surrounding tissue. To prevent this, thrombocytes in combination with soluble plasma components form a hemostatic thrombus which seals off the damage and has the effect of stopping bleeding. As soon as a lesion occurs on a vessel, the various cellular and biochemical mechanisms necessary for hemostasis are immediately set in motion. The endothelium also plays a central role in arterial hemostasis by regulation of the permeability for plasma lipoproteins, leukocyte adhesion and secretion of pro- and antithrombotic factors and vasoactive substances.

The endothelium forms the single-layered lining of the vessel wall which separates the blood stream from the thrombogenic structures of the subendothelium. In the event of endothelial damage to the vessel wall and the subendothelial matrix now lying open, in the context of hemostasis adhesion of latent thrombocytes circulating in the blood to the collagen now exposed takes place. This initial adhesion process is controlled by thrombocytic membrane glycoprotein receptors, the integrins, and results in a change in shape, inactivation of thrombocytes and release of constituents from the storage granules. During this process, the thrombocytic glycoprotein VI interacts directly with the exposed collagen and stabilizes the binding. GPVI, as the most important collagen receptor, not only mediates firmer binding directly to collagen, but also mediates activation of other receptors necessary for adhesion. After the adhesion, aggregation leading to an accumulation of thrombocytes in the thrombus follows as the next step in hemostasis.

Glycoprotein VI (GPVI), as a collagen receptor on the surface of thrombocytes, therefore plays a decisive role in the activation of blood platelets and is also a risk factor for myocardial infarctions. Thrombocytes without GPVI show no adhesion to collagen and the capacity for activation and the aggregation is significantly reduced.

The supply of blood to the tissue is no longer ensured due to the occurrence of such thrombi, so that ischemic states of the tissue lying distally to the thrombus may occur.

Cardiovascular diseases, such as e.g. angina or myocardial infarction, thus currently still make up approx. one third of all deaths worldwide. With these diseases, rapid reperfusion of the coronary arteries affected by ischemia is of extreme importance, in order to prevent damage to the myocardium.

As the blood flow in a coronary vessel is reduced, irreversible damage occurs to the myocytes, which causes the functional metabolism in the myocardium to stop, as a result of which cell destruction finally occurs due to necrosis and apoptosis.

As mentioned above, transluminal percutaneous angioplasty in combination with a stent implant is currently employed for re-establishing or therapy of normal coronary circulation. After implantation of the stent, free flow through the vessel is indeed ensured again, but the vascular endothelium which represents a barrier between the circulating blood cells and the subendothelial matrix under physiological conditions is still damaged. Adhesion of blood platelets and subsequent formation of thrombi and the resulting acute myocardial infarction are therefore a major complication after a stent implant.

On reperfusion of the region previously ischemic due to blockage of a vessel, this is supplied with oxygenated blood again, as a result of which on the one hand cell damage is limited, but this process is associated with continuing damage to the myocardium. Interventional therapy methods, such as percutaneous transluminal coronary angioplasty, coronary stent implantation, laser ablation angioplasty etc., or antithrombotic therapy with medicaments, such as thrombolysis and fibrinolysis, are currently employed for acute therapy of myocardial infarction. Both therapy methods work towards the same aim, namely the fastest possible re-opening of the occluded vessel and therefore the obligatory reperfusion of the ischemic tissue.

Since stents coated with medicaments which are released gradually after implantation are currently also employed, the re-endothelialization of the treated vessel is also delayed by the medicaments released, so that stent thrombosis is an extremely critical complication of this method.

### SUMMARY OF THE INVENTION

An object of the present invention is therefore to provide a novel agent for maintaining endothelial integrity for prevention of arteriosclerotic plaque erosion, with which the disadvantages of the prior art can be overcome.

According to the present invention, this object is achieved by a bispecific fusion protein which (a) comprises a first polypeptide which binds to collagen, and (b) a second polypeptide which binds to endothelial precursor cells.

The object on which the invention is based is achieved completely in this manner.

According to the invention, a “fusion protein” is understood as meaning a hybrid protein or an artificial protein which can be prepared in vitro and also in vivo by molecular biology or chemical processes known in the prior art.

Precursor (progenitor) cells are generally derivatives of an adult stem cell, and on the one hand have stem cell properties with respect to their capacity for regeneration, but on the other hand are fixed to their future functional region, this “fixing” still being reversible. Cells circulating in the blood which have the ability to differentiate into endothelial cells are accordingly called “endothelial precursor cells”. These endothelial precursor cells carry specific cell surface proteins and can therefore in turn be captured via polypeptides which bind to these cell surface proteins.

In the present case, “polypeptide” is understood as meaning any chain of at least two amino acids joined to one another; the term “polypeptide” therefore also includes proteins which, like polypeptides, are made up of several amino acids joined to one another. Sometimes also only complete molecules in a stable form are called “proteins”, whereas “polypeptides” or “peptides” are understood as meaning shorter amino acid chains without a stable 3-dimensional structure. However, since no clear boundary can be drawn between these terms, in the present case the term “polypeptides” also explicitly includes proteins according to the definition.

The fusion protein can therefore be prepared e.g. by conjugation of two (or more) polypeptides by means of one or more chemical reagents or by recombinant DNA technologies. On the other hand there is the possibility of generating the fusion protein by using conventional expression vectors which code for the fusion protein according to the invention. These expression vectors are introduced into a suitable cell, which then produces the fusion protein.

The inventors of the present application have been able to demonstrate in their own studies that CD34⁺ stem cells can be recruited to exposed collagen surfaces with the fusion proteins according to the invention. Furthermore, the inventors of the present application have been able to demonstrate that by recruiting of the stem cells to the exposed collagen, it was possible to mature the stem cells into mature endothelial cells after a certain period of time, which led to re-endothelialization of the damaged tissue.

With the fusion proteins according to the invention it is consequently possible that the re-endothelialization and repair of damaged vessels or of any tissue which releases or exposes collagen on its surface due to damage or other influences can be treated by colonization with stem cells and maturation thereof into endothelial cells. As a result, it is possible to prevent the vessel-damaging reactions caused by a stent implant or by chemical agents, or to treat them successfully after they occur.

About 27 different collagens are currently known. At up to one quarter of the total weight of proteins, they make up the largest proportion of proteins in the body. Collagens are composed of in each case three identical or different alpha chains which are wound tightly around one another.

Endothelial precursor cells are a circulating cell population, derived from bone marrow, of large non-leukocytic cells which are evidently involved in the repair of vessels and in hemostasis. Using the bispecific construct according to the invention, it was possible to recruit stem cells to damaged human tissue under flow conditions.

One advantage of the fusion protein is furthermore that the substance can be administered directly e.g. via a balloon catheter, or can be co-incubated with a stem cell population before these cells are administered, without a difficult and expensive coating of the coronary stents being necessary. The present fusion protein therefore represents an extremely effective tool with which stem cells can be recruited to damaged vascular lesions, and therefore represents an effective therapeutic concept for treatment of arterosclerotic diseases.

According to one aspect of the invention, the collagen-binding first polypeptide is chosen from the group including collagen antibodies, collagen receptors or functional fragments thereof. In particular, the collagen receptor may be chosen from the group including thrombocytic glycoprotein VI (GPVI), discoidin domain receptor 1 (DDR-1), discoidin domain receptor 2 (DDR-2), or functional fragments thereof.

As already mentioned above, the receptor GPVI is the most important receptor of thrombocytes for collagen. GPVI makes aggregation, secretion, change in shape and activation of blood platelets possible. Human GPVI contains a signal sequence with 20 amino acids, an extracellular domain of 247 amino acids, and a transmembrane domain 21 amino acids long and a cytoplasmic tail 51 amino acids long.

The discoidin domain receptors 1 (DDR-1) and 2 (DDR-2) are receptor tyrosine kinases and are characterized by the discoidin domains in the extracellular region of the receptor. Discoidin domain receptors are made up of an extracellular discoidin domain, a transmembrane domain, a long juxtamembrane domain and an intracellular kinase domain. Their binding to collagen has been described in the prior art (see e.g. Vogel et al., “The discoidin domain receptor tyrosine kinases are activated by collagen”, Mol. Cell (1997) 1:13-23). DDR-1 comprises 913 amino acids, the extracellular domain comprising amino acids 19 to 416; DDR-2 comprises 855 amino acids, and amino acids 22 to 399 form the extracellular domain here.

The fusion protein according to the invention binds with the said receptors or receptor fragments as the first polypeptide on exposed collagen. Endothelial precursor cells, that is to say particular stem cells, are recruited to the exposed collagen via the second polypeptide contained in the fusion protein, and in particular by the endothelial precursor cells binding by their specific surface antigens, such as e.g. CD133, to the polypeptide of the fusion protein which recognizes the antigens. The stem cells recruited in this way mature into endothelial cells after a certain incubation period, and can thereby regenerate the damaged tissue, as a result of which collagen is no longer exposed and is therefore no longer thrombocytic.

According to another aspect, the first polypeptide has an extracellular portion of GPVI, an extracellular portion of DDR-1 or an extracellular portion of DDR-2, or functional fragments thereof, combined with an immunoglobulin Fc domain.

It is advantageous here that e.g. already soluble GPVI, which has been described previously in the prior art, see Massberg et al., “Soluble glycoprotein VI dimer inhibits platelet adhesion and aggregation to the injured vessel wall in vivo”, FASEB J. 2004; 18: 397-399, reference being made explicitly to this publication with respect to the preparation of soluble human GPVI, can be used. Soluble GPVI shows affinity for collagen only as the dimeric form in association with the immunoglobulin Fc domain. To generate this soluble GPVI, the extracellular contain of human GPVI was cloned and combined with the human immunoglobulin Fc domain. This GPVI-Fc protein (called soluble GPVI-FC in the following) can be expressed e.g. with the aid of adenoviruses via a human HeLa cell line. It was possible to demonstrate adhesion to collagen with this soluble GPVI-Fc both in vitro and in vivo.

It goes without saying for a person skilled in the art that to fulfill the function according to the invention the fusion protein, the complete or identical amino acid sequence of soluble GPVI does not necessarily have to be employed. Rather, the function according to the invention of the fusion protein is also fulfilled if the first polypeptide has a section or a sequence variant of soluble GPVI which, however, still exerts the binding function of GPVI in a possibly attenuated form. As is known, the proteinogenic amino acids are divided into four groups, namely into polar, non-polar, acidic and basic amino acids. The exchange of one polar amino acid for another polar amino acid, e.g. glycine for serine, as a rule leads to no or only a slight change in the biological activity of the corresponding protein, so that such an amino acid exchange leaves the function of the fusion protein according to the invention largely untouched. Against this background, the present invention also includes such a fusion protein which, as the first polypeptide, is a variant of soluble GPVI in which one or more amino acids of one of the said amino acid classes is exchanged for another amino acid of the same class. In this context, such a sequence variant is preferably homologous to the amino acid sequence of soluble GPVI to the extent of approx. 70%, more preferably to the extent of approx. 80% and most preferably to the extent of approx. 90 to 95%.

“Fc” means “fragment crystallizable”. This fragment is formed by papain cleavage of the IgG molecule, alongside the two Fab fragments. The Fc domain is made up of the paired C_{H}2 and C_{H}3 domains, including the hinge region, and contains the part of the immunoglobulin responsible for the dimerization function. Commercially obtainable human or mouse Fc-DNA, which either can be isolated from commercially obtainable cDNA libraries by PCR or are already cloned in plasmids, which in turn can be obtained commercially (e.g. obtainable from Invitrogen, San Diego, USA), can advantageously be used here.

It goes without saying that a fragment or a variant of the Fc domain can also be used without the function according to the invention of the first polypeptide being impaired, as long as the fragment or the variant still has the possibly attenuated dimerization function of an antibody; cf. the above descriptions of the fragment or the variant of GPVI, which apply similarly to the fragment or the variant of Fc.

In this context, a variant of the Fc domain or a synthetic Fc fragment which is mutated in the complement and Fc receptor binding region such that activation of the immune system is largely reduced and possibly even absent is preferably employed. Thus e.g. an Fc fragment in which a proline is exchanged for a serine at position 331 and the tetrapeptide Leu-Leu-Gly-Gly is exchanged for Ala-Ala-Ala-Ala at amino acid positions 234 to 237 by targeted mutagenesis can be employed.

According to another aspect of the invention, the first polypeptide has an amino acid sequence with SEQ ID NO:3, 5 or 7 from the attached sequence listing.

The amino acid sequence SEQ ID NO:3 represents the extracellular domain of human GPVI, the total sequence of which is reproduced in SEQ ID NO:1.

The amino acid sequence SEQ ID NO:5 represents the extracellular domain of human DDR-1, the total sequence of which is reproduced in SEQ ID NO:4, and the amino acid sequence SEQ ID NO:6 represents the sequence of DDR-2, the extracellular domain of which is shown in SEQ ID NO:7.

In this context, the fusion protein can contain a first polypeptide which is coded by a section of the nucleic acid molecule which has the nucleotide sequence SEQ ID NO:2 from the attached listing.

The nucleotide sequence SEQ ID NO:2 represents the nucleotide sequence coding for human GPVI.

It goes without saying that not only the nucleotide sequence SEQ ID NO:2 is suitable for preparation of the extracellular domain of the collagen receptors, but also variants thereof which code for the same polypeptide due to degeneration of the genetic code. It is thus known that the genetic code is degenerated since the number of possible codons is greater than the number of amino acids. For most amino acids there is more than one codon, so that e.g. arginine, leucine and serine is coded by up to six codons. As a rule, the third codon position can be exchanged to a limited degree or completely. Against this background, such a fusion protein in which the first polypeptide is coded by a nucleic acid molecule which deviates from nucleotide sequence SEQ ID NO:2 at individual nucleotide positions due to degeneration of the genetic code, but codes similarly for the extracellular domains of GPVI and DDR-1 or DDR-2, is provided. Preferably, such a variant shows approx. 70% homology to nucleotide sequence SEQ ID NO:2, more preferably approx. 80% homology and most preferably approx. 90 to 95% homology.

According to another aspect of the invention, the second polypeptide is an antibody directed against CD133, or functional fragments thereof.

The antigen CD133 is expressed on hematopoietic and endothelial precursor stem cells and on some epithelial cells. The antigen accordingly is a marker for these stem cells, and has been described adequately in the prior art (see e.g. Yin et al., “CD133: A novel marker for human hematopoetic stem and progenitor cells”, Blood (1997) 90:5002-5012). Via an antibody which recognizes this antigen, the stem cells, which in turn contain this antigen, can accordingly be recruited to the collagen by binding to the second polypeptide of the fusion protein.

Thus e.g. an anti-CD133 antibody, or functional fragments thereof, which is currently commercially obtainable, such as e.g. the CD133 antibody of Miltenyi Biotech (clone W6B3C1), Bergisch Gladbach, Germany or the CD133 antibody from Abcam Inc. (32AT1672), Cambridge, Great Britain, can be employed.

The antibody W6B3C1 was obtained by immunization of mice with the retinoblastoma cell line WERI-RB-1.

It goes without saying that any antibody directed against CD133, or functional fragments thereof, can be employed for the purpose of the present invention. By employing the appropriate antigen, it is possible to generate further novel anti-CD133 antibodies using the conventional techniques in the prior art (e.g. the hybridoma technique, see Köhler and Milstein, “Continuous cultures of fused cells secreting antibody of predefined specificity”, Nature (1975) 256:495-7).

In the present case, “functional fragments” of an antibody mean any antibody sections or parts which have the same function or binding specificity as the whole antibody from which they are derived.

It goes without saying that starting from mouse anti-CD133 antibodies known in the prior art, humanized antibodies can also initially be obtained, which are then employed in the fusion construct. Humanized antibodies are recombinant antibodies in which the sequences for the hypervariable regions (CDR) in human immunoglobulin genes are exchanged for the CDR of immunoglobulin genes of the mouse. The antigen specificity of a monoclonal antibody of the mouse is transferred to a human antibody by this humanization. A complete tolerance to these molecules can thereby be produced in the recipient organism, as a result of which a human anti-mouse antibody response and is avoided. Such antibodies are also called chimeric antibodies.

According to another aspect of the invention, a further peptide element which joins the first polypeptide to the second polypeptide can be provided in the fusion protein. The polypeptides can also be joined via a bridge or a linker by this means, the functionality of the two polypeptides, that is to say thus the specific recognition of the particular binding sites, being retained at the same time.

The invention furthermore relates to a pharmaceutical and/or diagnostic composition which comprises the fusion protein as claimed in one of claims 1 to 6, and at least one pharmaceutically acceptable carrier and optionally further pharmaceutically and/or diagnostically active substances.

Diagnostically and pharmaceutically acceptable carriers with optionally further additives are generally known in the prior art and are described e.g. in the article by Kibbe A., Handbook of Pharmaceutical Excipients, Third Edition, American Pharmaceutical Association and Pharmaceutical Press 2000. According to the invention, additives include any compound or composition which are advantageous for a diagnostic or therapeutic use of the composition, under which fall salts, binders and further substances conventionally used in connection with the formulation of medicaments.

The invention furthermore relates to methods for using the fusion protein for treating or preventing lesions of vessels and tissues in a mammalian subject, and the invention in particular relates to a method for treating or preventing lesions of vessels in a mammalian subject, preferably a human subject, comprising administering the fusion protein of the invention to the mammalian subject in need thereof; the vessels and/or tissue of the mammalian subject may be chosen from the group including coronary vessels, vessels which supply the brain, vessels which supply the extremities, connective tissue, bone, and any vessel or tissue which contains collagen.

A composition prepared according to the invention which comprises the fusion protein according to the invention provides an extremely effective tool for treatment of diseases of which the cause is lesion of vessels or tissues with which thrombogenic subendothelium is exposed as a consequence, which can lead to formation of thrombi.

According to one aspect of the invention, the bispecific fusion protein is administered via a balloon catheter.

Alternatively, the bispecific fusion protein may be co-incubated with a stem cell solution before administrating the cells to a mammalian subject.

This has the advantage that difficult and expensive coating processes on coronary stents are avoided.

The invention furthermore relates to a process for the preparation of a fusion protein with the following steps: (a) provision of a soluble form of glycoprotein VI (GPVI) and of an antibody directed against CD133; (b) modification of the amino groups of GPVI and of the antibody with a crosslinking agent; (c) reduction of GPVI; and (d) conjugation of the reduced GPVI with the antibody modified in step (b).

In particular, it is preferable for the crosslinking agent in the process according to the invention to be SPDP N-succinimidyl 3-(2-pyridyldithio)-propionate).

Alternatively, any other crosslinking agent or coupling process known in the prior art can also be employed, such as e.g. bonding via a thioether crosslinking agent, or via recombinant DNA technology.

A fusion protein which is suitable for a diagnostic/therapeutic purpose or use can be provided by the process according to the invention.

It goes without saying that the abovementioned features and the features still to be specified further in the following are possible not only in the particular combination stated but also in other variations or by themselves without leaving the context of the present invention.

### BRIEF DESCRIPTION OF THE FIGURES

The patent or application file contains at least one drawing executed in color. Copies of this patent or patent application publication with color drawings will be provided by the Office upon request and payment of the necessary fee.

The invention is explained in more detail in the following example and in the figures. The figures show

FIG. 1 a diagram of an embodiment of the bispecific construct according to the invention, which is directed against collagen and the stem cell antigen CD133.

FIG. 2 Recruiting of EPCs to exposed collagen by a specific GPVI/CD133 construct made up of the soluble collagen receptor GPVI and an antibody directed against CD133 leads to development of endothelial cells in vitro: a) static adhesion assay; b) dynamic assay; c) formation of endothelial colonies; d) marker expression of CD31 and CD146; e) expression of vWF/endoglin; f) detection of Weibel-Palade bodies in an electron microscope; g) specific intensified adhesion of the EPCs, mediated by GPVI−CD133, to immobilized collagen compared with fibronectin; h) more effective recruiting of EPCs to collagen by GPVI−CD133 than CXCL7; and

FIG. 3 The GPVI−CD133 construct recruits EPCs to vessel lesions in vivo and intensifies the repair of tissue integrity: a), b) damage to the carotid artery of test animals and injection of EPCs with DCF staining; c) histology sections analyzed by two-photon microscopy and stained with DCF; d) in situ hybridization of histology sections with a human alu sequence; e) HE staining of endothelial cells; in situ hybridization with an alu sequence; f) GPVI−CD133 has the effect of a significantly decreased intima/media ratio.

### DESCRIPTION OF PREFERRED EMBODIMENTS

### Example 1

### Preparation of a Bispecific Protein/Monoclonal Antibody Construct for Recruiting of Bone Marrow Stem Cells to Vessel Lesions

### Material and Methods

### Reagents

Biocoll separating solution was obtained commercially from Biochrom AG (Berlin, Germany), and EBM as “BulletKit” (EGM) from Cambrex Bio Science (East Rutherford, N.J.). Collagen I, collagen III, laminin, vitronectin, fibrinogen and fibronectin were obtained commercially from BD Sciences (Heidelberg, Germany), human VEGF from PeproTech Inc. (Rocky Hill, N.J.), and the primary mouse antibody anti-vWF and the phalloidin-AlexaFluor 488 from Chemicon (Temecula, Calif.). DAPI, the Cy3-labeled secondary antibody (goat anti-mouse) and the “Celltracker Vybrand DiD” was obtained commercially from Molecular Probes/Invitrogen GmbH (Karlsruhe, Germany).

Isolation and Culturing of CD34⁺ Cells and CD133⁺ Cells

Human CD34⁺ cells and CD133⁺ cells were isolated from human umbilical cord blood and cultured as described by Lang, et al. (“Transplantation of a combination of CD133+ and CD34+ selected progenitor cells from alternative donors”, British Journal of Haematology 2004; 124: 72-79). The donor cells were mobilized by administration of 1×10 μg/kg of granulocyte stimulating facto (G-CSF) for 5 days and harvested by 1 to 3 leukapheresis processes. The selection of the precursor cells with microbeads coated with anti-CD34⁺ or anti-CD133⁺ was performed with the automated CLINIMACS device (Miltenyi Biotec, Bergisch Gladbach, Germany). Before and after separation of the cells, the cell populations were stained with anti-CD34⁺ anti-CD133⁺, anti-CD3⁺, antiDC19⁺ and anti-CD45⁺ antibodies and analyzed by fluorescence-activated cell sorting equipment (FACS) with FACSCalibur instruments (Becton-Dickinson, Heidelberg, Germany).

Preparation of a GPVI−CD133⁺ mAB Construct

In order to effect the adhesion of stem cells to exposed collagen, a bispecific construct (fusion protein) was prepared. For this, soluble GPVI-Fc and a monoclonal antibody against CD133 was used. Soluble GPVI was prepared as described previously, in this context see Massberg et al., see above, reference being made explicitly to this publication for preparation of the soluble GPVI construct. Briefly, the extracellular domain of GPVI was fused to the human Fc domain. For this, Fc was amplified from a human heart cDNA library (Clontech, Palo Alto, Calif., USA). The primer pairs and the conditions for the polymerase chain reaction are to be found in the cited publication of Massberg et al. The PCR fragment was cloned via NotI/HindIII into the plasmid pADTrack CMV. For cloning of the extracellular domain of human GPVI, total RNA was isolated from cultured megakaryocytes (RNeasy Mini Kit, Qiagen, Hilden, Germany). After a reverse transcription, 100 ng of the cDNA generated were employed as the template for the PCR amplification of human GPVI (for primers and PCR conditions, see the publication cited). The PCR fragment was cloned into the plasmid pADTrack CMV Fc via BglII/NotI, as a result of which a plasmid was obtained which contained the human extracellular domain of GPVI, fused to the human Fc domain, including a specific hinge region.

The CD133-reactive monoclonal antibody (mAB) W6B3C1 was generated by immunization of 6 week-old female Balb/c mice (Charles River WIGA, Sulzfeld, Germany) with the retinoblastoma cell line WERI-RB-1. The specificity of the monoclonal antibody for CD133 was confirmed at the 7th International Leukocyte Conference in England (see Bühring et al., “CD133 Cluster Report.” In: Leucocyte Typing VII. White Cell Differentiation Antigens, Mason D et al., (eds.), Oxford University Press, Oxford, 2002, pages 622-623).

For conjugation of the two proteins, the heterobifunctional reagent SPDP (N-succinimidyl 3-(2-pyridyldithio)-propionate) was employed in accordance with the method of Carlsson et al., “Protein Thiolation and Reversible Protein-Protein Conjugation”, Biochem. J. 173:723 (1978). For this, the amino groups of the two proteins were modified by means of SPDP. The modified GPVI protein was reduced with DTT (dithiothreitol) and conjugated with the non-reduced, SPDP-modified CD133 antibody. The conjugation mixture was purified by gel filtration over a Superdex S200 column.

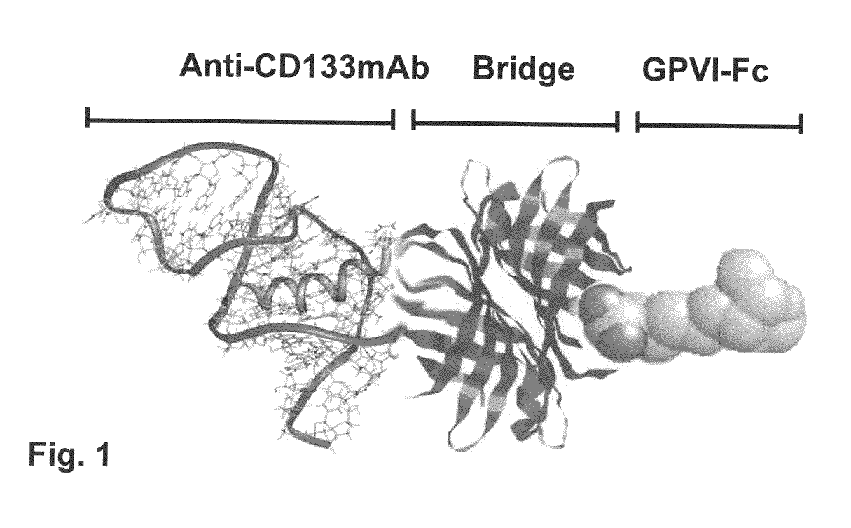

A diagram of the bispecific construct obtained in this way is shown in FIG. 1.

Static and Dynamic Adhesion Assays

Static adhesion. In order to determine the adhesion of the precursor cells to various extracellular matrix proteins with or without the fusion protein under static conditions, 96-well plates were coated overnight with collagen I, fibrinogen, fibronectin or vitronectin (in each case 10 μg/ml). In further experiments the 96-well plates coated with collagen I were pre-incubated with the fusion protein (10 μg/ml) for one hour. The individual components of the construct together or the individual components alone served as a negative control. The precursor cells were then added and incubation was carried out for one hour. After three careful washing steps with Tyrode's buffer, the remaining adhering precursor cells were counted by means of phase contrast microscopy.

Dynamic adhesion. For this, glass microscope slides were coated with collagen I (10 μg/ml) (see Langer et al., “ADAM 15 is an adhesion receptor for platelet GPIIb-IIIa and induces platelet activation”, Thromb. Haemost. 2005; 94:555-561) and inserted into a flow chamber (Oligene, Berlin, Germany). The fusion protein (10 μg/ml) was then added to the collagen surface over 30 min. Experiments with the individual components together or the individual components alone again served as a control. The perfusion was performed with stem cells which in Tyrode's-HEPES buffer (HEPES 2.5 mmol/l; NaCl 150 mmol/l; KCl 1 mmol/l; NaHCO₃ 2.5 mmol/l; NaH₂PO₄ 0.36 mmol/l; glucose 5.5 mmol/l; BSA 1 mg/ml, pH 7.4, supplemented with CaCl₂ 1 mmol/l; MgCl₂ 1 mmol/l; each from Sigma, Taufkirchen, Germany) with a shear rate of 2,000 s⁻¹. All the experiments were recorded on video in real time and evaluated off-line.

Colony Formation Assay and Flow Cytometry

CD34⁺ precursor cells were sown on human collagen I under the following various conditions: with or without addition of the GPVI−CD133 construct (10 μg/ml), the two individual components of the construct (negative control), fibronectin (Becton Dickinson, Heidelberg, Germany) as a positive control. The cells were in each case cultured for several days in growth medium for endothelial cells MV2 with 5% heat-inactivated fetal calf serum, 5.0 ng/ml of epidermal growth factor, 0.2 μg/ml of hydrocortisone, 0.5 μg/ml of vascular endothelial growth factor, 10 ng/ml of basic fibroblast factor, 20 ng/ml of R3 insulin-like growth factor 1 and 1 μg/ml of ascorbic acid (PromoCell, Heidelberg, Germany). After 48 hours the non-adhering cells were removed. Endothelial colony-forming units were counted on day 4 (number of colonies/10⁶ cells). The cells were washed and resuspended in PBS, incubated for 15 min with Polyglobin (Bayer Vital; Leverkusen, Germany), washed and then incubated with FITC-labeled antibodies against CD31 (clone 5.6; Beckman Coulter, Krefeld Germany) and CD164 (clone 128018; R&D Systems Wiesbaden, Germany) at room temperature for 30 min. After a further washing step, the cells were analyzed with an FACSCanto flow cytometer (Becton Dickinson, Heidelberg, Germany).

Transmission Electron Microscopy and Immunofluorescence Microscopy

Endothelial precursor cells (EPC) (2×10⁸/ml) were incubated in culture medium MV 2 (PromoCell) for eight days in wells coated with GPVI−CD133⁺ mAB. Phase contrast controls were moreover performed daily. The cells were then fixed in Karnovsky's solution, after-fixed in osmium tetroxide and embedded in glycidyl ether, before the microscopy was performed.

For the immunofluorescence microscopy, the cells were additionally incubated with fluorescence-labeled antibodies. Between each incubation step the cells were washed carefully with PBS. The stem cells were fixed in 2% formaldehyde solution for 20 minutes. The cells were then washed with 3% glycine and incubated for 30 minutes with PBS which contained a primary anti-vWF antibody (human; 5 μg/ml). Non-specific binding was prevented with bovine serum albumin (3%, one hour). Thereafter, a secondary antibody (goat anti-mouse; 5 μg/ml) was added for a further 30 minutes. Rhodamine phalloidin (5 μg/ml; detection of the cytoskeleton) and DAPI (5 μg/ml; detection of the cell nucleus) were furthermore added for 30 minutes. The samples were analyzed by means of a standard immunofluorescence microscopy.

Ligature of the Carotid Artery and Investigation of the EPC Adhesion by Intravital Microscopy

In order to investigate the effect of the GPVI−CD133 construct on recruiting of progenitor cells in vivo, an intravital microscopy was carried out as already described elsewhere (see Massberg et al., “A critical role of platelet adhesion in the initiation of atherosclerotic lesion formation”, J. Exp. Med. 196: 887-896 (2002)). Before the experiments, the EPCs were stained with 5-carboxyfluorescein diacetate succinimidyl ester (DCF) and incubated with the GPVI−CD133 construct (10 μg/ml) or the two individual components of the construct (in each case 10 μg/ml) for 30 min. Wild-type C57BL6/J mice (Charles River Laboratories) were anesthetized by intraperitoneal injection with midazolam (5 mg/kg of body weight); Ratiopharm), medetomidin (0.5 mg/kg of body weight; Pfizer) and fentayl (0.05 mg/kg of body weight; CuraMed/Pharam GmbH). Polyehtylene catheters (Portex) were implanted into the right-hand jugular veins and fluorescent EPCs (5×10⁵/ml) were injected intravenously. The right-hand carotid arteries were exposed and ligated energetically close to the carotid fork for five minutes in order to induce damage to the vessel. Before and after the damage to the vessel, the interaction of the fluorescent EPCs with the damaged vessel wall was rendered visible by in situ in vivo video microscopy of the right-hand carotid artery using a Zeiss Axiotech microscope (20× water immersion lens, W 20×/0.5; Carl Zeiss MicroImaging, Inc.) with a 100-W HBO mercury lamp for the epi-illumination. Bound EPCs were defined as cells which built up an initial contact with the vessel wall, followed by a slow surface translocation with a speed significantly slower than the average speed, or by a firm adhesion. The number of adhering EPCs were determined by counting the cells which did not move or did not detach themselves from the endothelium surface within 10 s. Their number is stated as cells/mm² of endothelium surface.

Two Photon Microscopy

The two-photon microscopy was carried out substantially as already described by van Zandvoort et al., “Two-photon microscopy for imaging of the atherosclerotic vascular wall: a proof of concept study”, J. Vasc. Res. 41: 54-63 (2004). Briefly, the mice were sacrificed after the intravital microscopy, the carotid arteries were carefully removed, washed with PBS and embedded in paraffin and 4 μm sections were prepared. The sections were then stained and analyzed with a BioRad 2100MP by the two-photon laser scanning microscopy (TPLSM) method.

Ex Vivo Investigation of the EPC Adhesion on Damaged Vessels from Pigs

After isolation, the stem cells were labeled with Vybrant DiD for 20 minutes and resuspended in EBM medium. Human veins were added in an ex-vivo flow in which the vessel was surrounded by medium for nutrient reasons. The vessels were damaged by means of a balloon catheter and then coated with the GPVI−CD133 mAb construct for 30 minutes. EPCs were then led through the veins for two hours in order to make adhesion of the cells to the damaged region of the vessels possible. In order to test the stability of the adhesion under natural physiological shear stress, the veins were then washed thoroughly with EBM with a high shear rate at 37° C. for 24 hours. Thereafter, the vessels were removed from the bioreactor, fixed in 4% PFA for 24 hours, and the cell recruiting was analyzed by in situ hybridization.

In Vivo Investigation of the Re-Endothelialization of Damaged Vessels

Wild-type C57BL6/J mice were treated in a similar manner to the protocol for investigation of the in vivo adhesion (see above). EPCs (5×10⁵/ml) which had been treated with the GPVI−CD133 construct (10 μg/ml9 or the two individual components of the construct together (in each case 10 μg/ml) for 2 hours, the wounds of the right-hand jugular veins were closed; the animals subsequently remained alive. After two weeks the animals were sacrificed and samples were removed from the carotid artery. Regenerating endothelial cells were investigated by hematoxylin-eosin (HE) staining. An elastica-von Giesson staining was additionally performed. In order to distinguish between local regeneration mechanisms and the healing induced by the human EPCs, in situ hybridizations were carried out using an alu sequence specific for human cells.

Immunohistochemistry of Paraffin Sections

Immunohistochemistry was carried out using paraffin sections from mouse vessels. The microscope slides with the sections were deparaffinized with xylene (Carl Roth GmbH, Karlsruhe, Germany) and rehydrated again with descending concentrations of ethanol: 100%, 90%, 70%, 50%. The microscope slides were then washed thoroughly with PBS. Thereafter, in each case 20-minute permeabilization and blocking steps with PBS, which contained 0.1% Triton® X-100 (Fluka Chemie, Buchs, Switzerland) and 1% BSA (bovine serum albumin) solution (Sigma Aldrich, St, Louis, USA) followed. The microscope slides were then incubated with the primary antibody anti-vWF (2.5 μg/ml) (Chemicon, Temecula, USA) at 4° C. for 12 hours. Thereafter, the secondary goat anti-rabbit antibody (5 μg/ml) (Molecular Probes/Invitrogen, Karlsruhe, Germany) and 0.1 μg/ml of DAPI (Carl Roth GmbH, Karlsruhe Germany) were added at room temperature for a further 120 min. The microscope slides were washed thoroughly with PBS, washed off with distilled water, dried and covered with Kaiser's gelatin (Merck, Darmstadt, Germany) and analyzed.

Determination of the Neointima Formation

Male NOD/SCID mice were treated in accordance with a protocol which is similar to that described previously (see under “Ligature of the carotid artery”). Instead of the carotid artery ligature, damage was brought about by means of a wire. After the injection of EPCs (5×10⁵/ml) which had been treated beforehand for 30 min with the GPVI−CD133 construct (10 μg/ml) or with the two individual components of the construct together (in each case 10 μg/ml) into the tail vein, the wounds were closed and the animals were kept alive. After 14 or after 21 days the animals were sacrificed and the carotid artery samples were removed. These were embedded in paraffin blocks and cut into 5 μm sections from the proximal to the distal end. Ten sections downwards of the carotid fork were employed for the quantification or the plaque formation. The neointima formation was determined in cross-section using imaging analysis software (Zeiss). The neointima was determined for each animal as the difference between the region demarcated by the internal elastic lamina and the lumen region. The media was determined in a similar manner, and in particular as the difference between the region demarcated by the internal elastic lamina and that of the outer elastic lamina. The results are presented as neointima divided by media (intima/media ratio).

Determination of the Vascular Resistance Index by Duplex Sonography

The animals were anesthetized and the carotid arteries were rendered visible by means of duplex sonography as described previously (see Massberg et al., “A critical role of platelet adhesion in the initiation of atherosclerotic lesion formation”, J. Exp. Med. 196:887-896 (2002)). Briefly, the maximum systolic flow rate V_{sys} and the endodiastolic flow rate V_{dia} was determined. The resistance index of the carotid artery was determined as the difference between V_{sys} and V_{dia} divided by V_{sys}.

Presentation of the Data and Statistics

Comparisons between the group means were performed using ANOVA analysis or the Student's t-test. The data are presented as means±standard deviation. P<0.05 was regarded as statistically significant.

### Results

Using human stem cells derived from bone marrow, the adhesion of EPCs to immobilized collagen I was first investigated in a static adhesion assay and under arterial shear conditions in a flow chamber model.

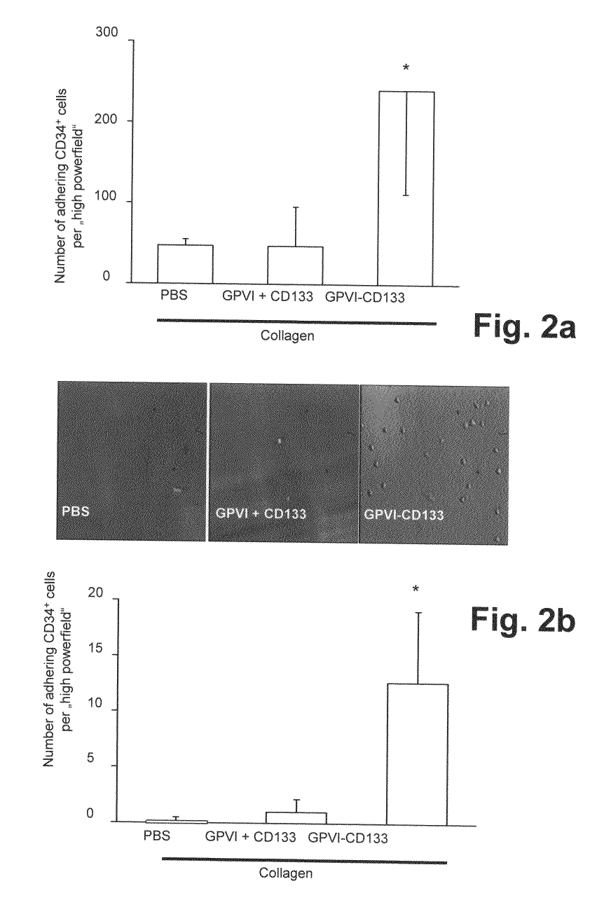

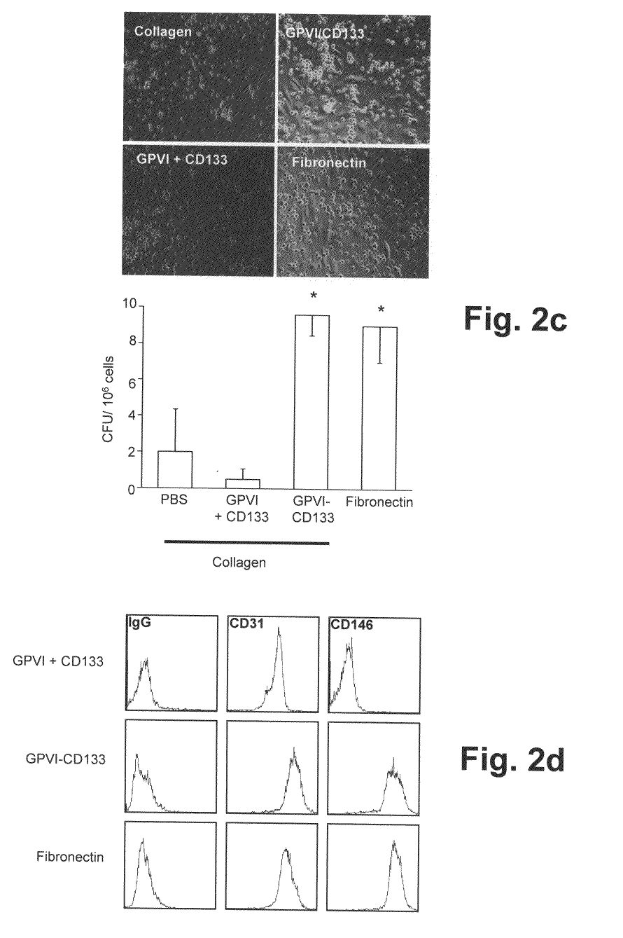

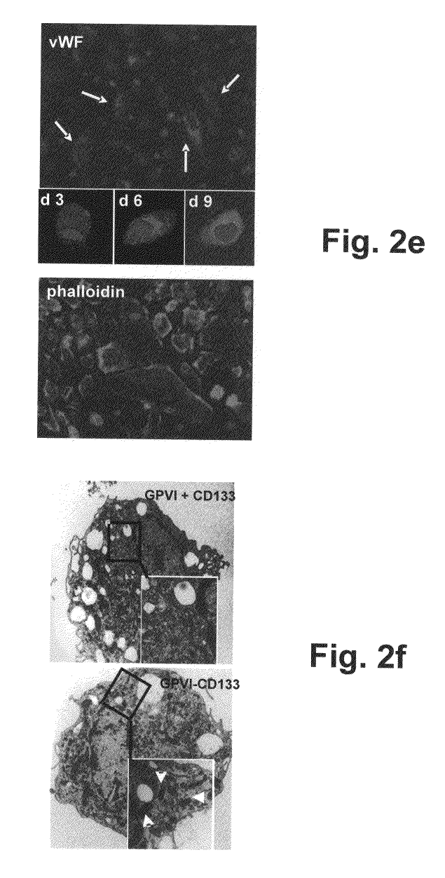

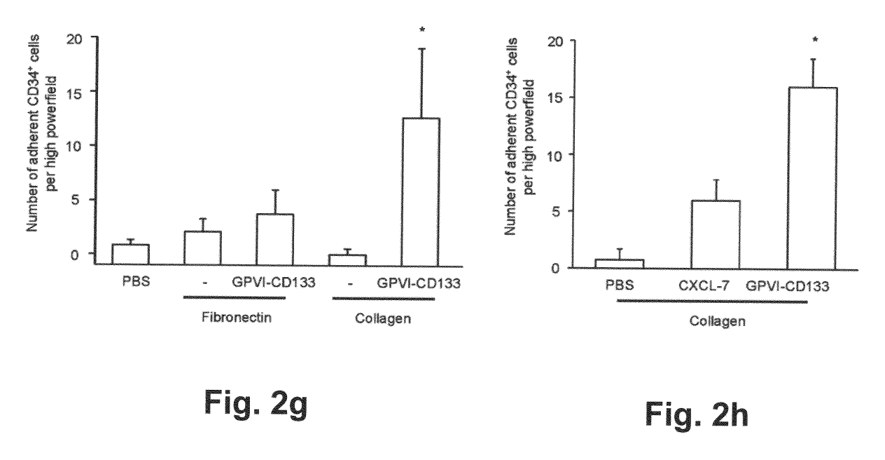

In the static adhesion assay, 96-well plates were coated with collagen I and incubated with the product described (10 μg/ml) for one hour. EPCs (CD34+ cells) were then added, incubation was carried out for 60 minutes and washing was carried out with PBS. After incubation of the collagen surface with the GPVI−CD133 construct (“GPVI−CD133”, 10 μg/ml), the adhesion was intensified 5-fold compared with collagen alone (see FIG. 2a; static model) and 10-fold in the flow chamber model (2,000 sec⁻¹) (FIG. 2b). No increase in the adhesion was to be observed when the two individual components of the construct were employed (in each case 10 μg/ml) The average and the standard deviation of 4 different experiments is shown. * means p=0.021 in d FIG. 2a and p=0.025 in FIG. 2b. This means that the construct is even more efficient under physiological flow conditions. Furthermore, it was possible to demonstrate in further experiments that the increased adhesion of the EPCs achieved by the GPVI−CD133 construct was specific for immobilized collagen compared with fibronectin (see FIG. 2g).

In all the figures the use of the construct is designated by “GPVI−CD133”, and the use of the individual components together is designated by “GPVI+CD133”.

It has recently been demonstrated that the chemokine CXCL7 can significantly increase chemotaxis and the adhesion of EPCs to components of the extracellular matrix. In this respect, it was possible to demonstrate in further experiments that the GPVI−CD133 construct can even more effectively have the effect of recruiting of the EPCs to immobilized collagen than CXCL7 (see FIG. 2h).

After the EPCs are bound, they are integrated into the endothelial layer, in order to contribute towards repairing the vessel integrity. It was therefore demonstrated in subsequent experiments that after use of the construct, the cells do not lose their ability to differentiate into endothelial cells. Furthermore, it was possible to observe a rapid change in morphology away from the small, roundish appearance of the EPCs into a rather endothelial cell shape after exposure to the construct. After incubation with the construct beyond 4 days, the potential of the EPCs to form endothelial colonies was increased significantly compared with the same experiments which were carried out with the individual components (negative control), and similarly to the positive control fibronectin (see FIG. 2c; number of colonies/10⁶ of cells employed). The average ± standard deviation of 3 to 5 independent experiments is shown. * corresponds to p=9.001.

Furthermore, it was possible to demonstrate with the flow cytometry that developing cells are positive for the cell markers CD31 and CD146, which represent endothelial surface markers (see FIG. 2d). It was furthermore possible to stain the cells positively for the markers vWF/endoglin and phalloidin, which represent markers of mature endothelial cells. Detection was carried out via standard or confocal immunofluorescence microscopy (see FIG. 2e). It was furthermore possible to detect unambiguously Weibel-Palade bodies in transmission electron microscopy after incubation with the construct for 8 days, a typical feature of mature endothelial cells (FIG. 2f; shown with ←, ≅300 nm×60 nm; magnification×80,000). No Weibel-Palade bodies were to be found in untreated CD34⁺.

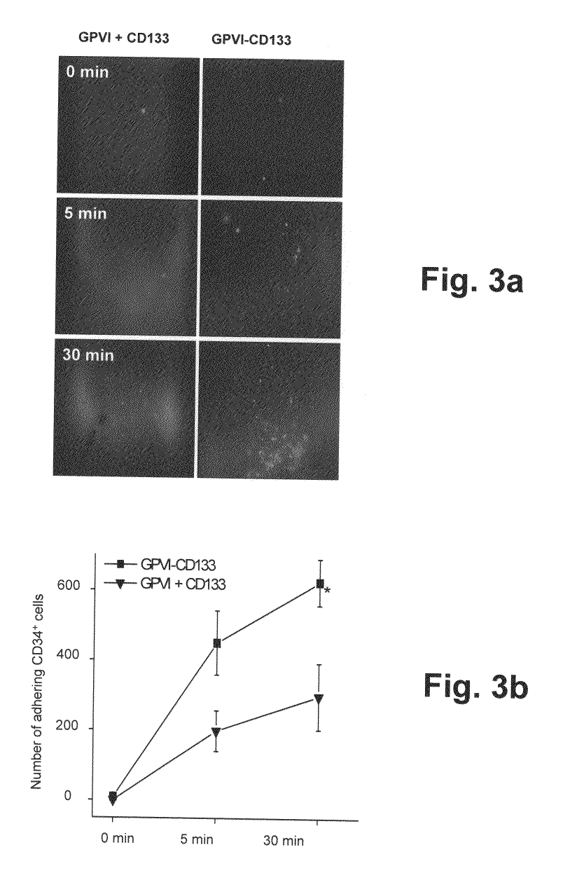

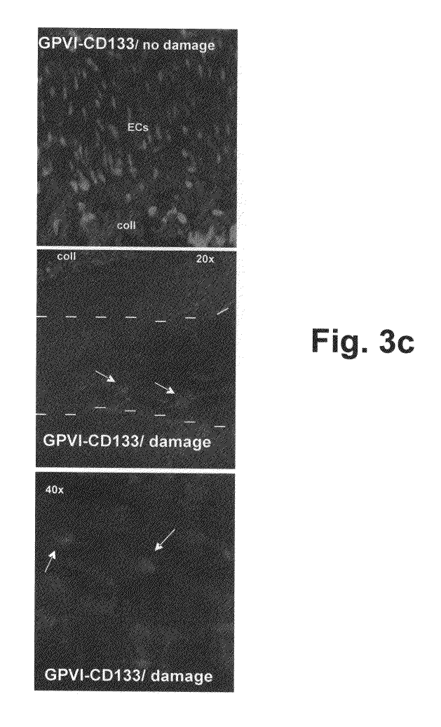

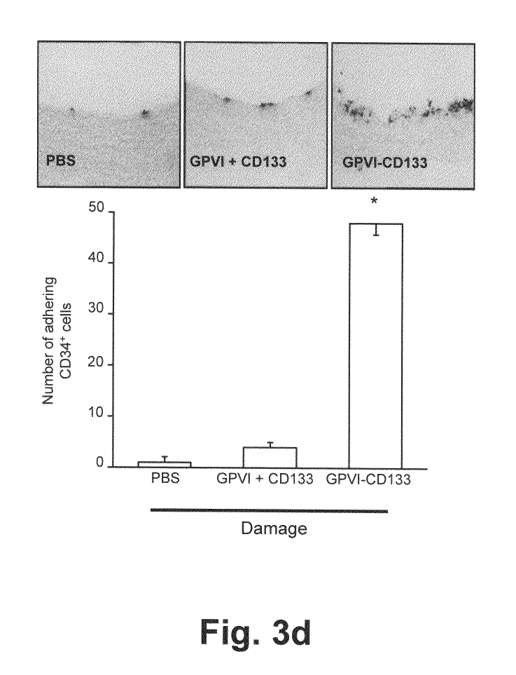

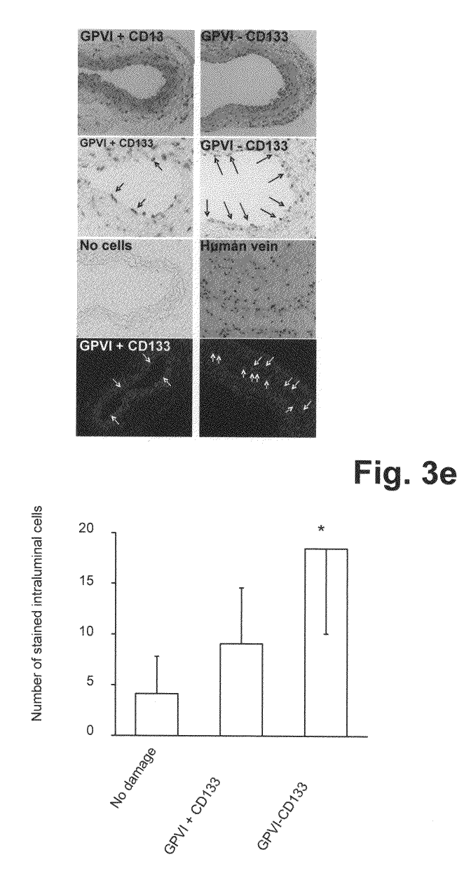

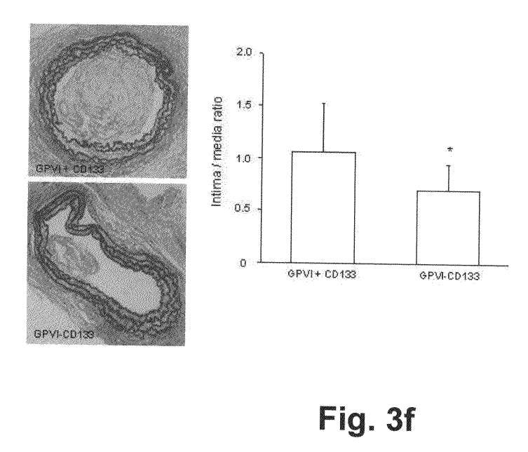

In order to confirm these results in vivo, an in vivo fluorescence microscopy and a mouse model with a damaged carotid artery was employed. Before energetic damage to the left carotid artery, EPS stained with DCF were injected via the right-hand jugular vein and the EPC adhesion was investigated before, after 5 min and after 30 min after causing the damage. The number of adhering EPCs was increased significantly if the cells were incubated beforehand with the GPVI−CD133 construct (“GPVI−CD133”, 10 μg/ml) compared with the individual components of the construct (“GPVI+CD133”, in each case 10 μg/ml) alone (see FIG. 3a, b). * means p=0.038 (firm adhesion), p=0.025 (transient adhesion).

After these investigations, the carotid arteries were removed and examined by means of two-photon microscopy. An obvious accumulation of green (DCF-stained) cells with a red nucleus was to be observed in the region of the denudation of the luminal side of the elastica interna (FIG. 3c).

In order to apply these results to a system comparable to humans, an ex vivo flow model was employed. For this, the vessels of pigs were damaged with a balloon catheter before the use of EPC and after perfusion for 2 hours. The vessels were then fixed and the recruiting of cells was investigated by in situ hybridization with a sequence specific for humans. It was possible to increase the recruiting of the stem cells significantly by the use of the GPVI−CD133 construct, compared with undamaged vessels (approximately 50-fold, not shown), with damaged vessels in which the construct was not employed (approximately 25-fold), or if the two components of the construct were employed alone (approximately 10-fold) (FIG. 3d). * means p<0.001 compared with the two individual components of the construct.

After exposure of the damaged mouse arteries to EPCs which had been treated with the bispecific construct, but not after exposure to the two individual components alone, over a period of eight days ex vivo (data not shown) or over 14 days in vivo, a production of endothelial cells was to be observed (FIG. 3e; HE staining). In order to distinguish between the effects caused by the cells administered and the effects caused by local regeneration mechanisms, immunodeficient NOD/SCID mice were treated with human EPCs. Hybridizations were then carried out in situ using an Alu probe. This specific Alu probe corresponds to the consensus sequence of human Alu repeats and makes a definitive detection of human cells in xenotransplants possible. For this, the mice were sacrificed 14 days after the damage caused to the carotid artery and after administration of cells. Intraluminal cells which proved to be positive in the staining were determined as cells derived from human cells. These results demonstrate that the neoendothelialization of vessel lesions essentially originated from externally injected EPCs.

In order furthermore to estimate the functional significance of GPVI−CD133 for vessel regeneration in vivo, the formation of neointima after damage caused by a wire was investigated. Two weeks after the damage was induced, a tendency in the direction of a reduced intima/media ratio and a reduced vessel resistance index was observed, without statistical significance, which was determined by duplex sonography (data not shown). It is striking that the administration of GPVI−CD133 resulted in a significantly reduced intima/media ratio 3 weeks after damage to the carotid artery was induced, which indicates the desired effect in vessel regeneration (See FIG. 3f). In these experiments also, again either the construct (GPVI−CD133) or the individual components together was administered (GPVI+CD133). In the diagram of FIG. 3f, “*” means p=0.03 compared with the control; n=5-6; 10 sections were analyzed per animal.

Summarizing, the inventors were therefore able to demonstrate that with the fusion protein according to the invention (also called “construct” above and below) it was possible for EPCs (that is to say CD34+ stem cells) to be accumulated on exposed collagen surfaces and damaged vessels in vitro, in vivo and in human vessels. The inventors were furthermore able to demonstrate that a longer incubation of the stem cells with the fusion protein the differentiation into mature endothelial cells can be achieved in vitro.

For a possible therapy of damaged vessels/tissue, this means that the fusion protein or variants derived therefrom can be inserted into the corresponding vessels e.g. via a catheter, or is co-incubated with stem cells before administration of these.

The results demonstrate that by means of the fusion protein according to the invention it is possible to capture circulating endothelial precursor cells on collagen-rich vessel lesions, which it has been possible to demonstrate both by in vitro and by in vivo experiments. The fusion protein moreover increased the differentiation of endothelial precursor cells (EPCs) into endothelial cells and increases the re-endothelialization of vessel lesions.

1. A bispecific fusion protein, comprising:
(a) a first polypeptide which binds to collagen, and
(b) a second polypeptide which binds to endothelial precursor cells.

2. The fusion protein as claimed in claim 1, wherein the first polypeptide comprises a peptide which is chosen from the group including collagen antibodies, collagen receptors or functional fragments thereof.

3. The fusion protein as claimed in claim 1, wherein the first polypeptide comprises a collagen receptor, which is chosen from the group including thrombocytic glycoprotein VI (GPVI), discoidin domain receptor 1 (DDR-1), discoidin domain receptor 2 (DDR-2), or functional fragments thereof.

4. The fusion protein as claimed in claim 1, wherein the first polypeptide has an extracellular portion of GPVI, an extracellular portion of DDR-1, an extracellular portion of DDR-2, or functional fragments thereof, combined with a dimerizing polypeptide.

5. The fusion protein as claimed in claim 1, wherein the first polypeptide has an extracellular portion of GPVI, an extracellular portion of DDR-1, an extracellular portion of DDR-2, or functional fragments thereof, combined with a dimerizing polypeptide having an Fc domain of an immunoglobulin or a fragment or a variant thereof which has the dimerization function of the Fc domain.

6. The fusion protein as claimed in claim 1, wherein the first polypeptide has the amino acid sequence SEQ ID NO:3, 5 or 7 from the attached sequence listing.

7. The fusion protein as claimed in claim 1, wherein the second polypeptide binds to the antigen CD133.

8. The fusion protein as claimed in claim 1, wherein the second polypeptide is an antibody directed against CD133, or functional fragments thereof.

9. A nucleic acid molecule which encodes the fusion protein as claimed in claim 1.

10. A pharmaceutical and/or diagnostic composition which comprises a bispecific fusion protein, comprising (a) a first polypeptide which binds to collagen, and (b) a second polypeptide which binds to endothelial precursor cells, and at least one pharmaceutically acceptable carrier and optionally further pharmaceutically and/or diagnostically active substances.

11. The pharmaceutical and/or diagnostic composition of claim 10, wherein the first polypeptide that binds to collagen is an extracellular portion of GPVI and the second polypeptide is an antibody that binds to CD133, combined with a dimerizing polypeptide having an Fc domain of an immunoglobulin or a fragment or a variant thereof which has the dimerization function of the Fc domain.

12. A method for using a bispecific fusion protein, the fusion protein comprising (a) a first polypeptide which binds to collagen, and (b) a second polypeptide which binds to endothelial precursor cells, for treating or preventing lesions of tissues and vessels where collagen is exposed.

13. The method of claim 12, comprising the step of administering to a mammalian subject in need thereof a therapeutically effective amount of the fusion protein.

14. The method as claimed in claim 12, wherein it is employed for re-endothelialization of vessel lesions.

15. The method as claimed in claim 12, wherein the vessels and/or tissue are chosen from the group including coronary vessels, vessels which supply the brain, vessels which supply the extremities, connective tissue, bone, and any vessel or tissue which contains collagen.

16. The method as claimed in claim 12, wherein the bispecific fusion protein is administered via a balloon catheter.

17. A process for the preparation of a fusion protein with the following steps:
(a) provision (i) of a polypeptide which is chosen from the group including: a soluble form of glycoprotein VI (GPVI), a soluble form of discoidin domain receptor 1 (DDR-1), or a soluble form of discoidin domain receptor 2 (DDR-2), and (ii) an antibody directed against CD133;
(b) modification of the amino groups of GPVI, DDR-1 or DDR-2, and of the antibody with a crosslinking agent;
(c) reduction of GPVI, DDR-1, or DDR-2; and
(d) conjugation of the reduced GPVI, DDR-1 or DDR-2 with the antibody modified in step (b).
